(19) United States
(12) Patent Application Publication
(10) Pub. No.: US 2026/0215939 A1
(43) Pub. Date: Jul. 30, 2026

(54) DILATANCY IN AN ADJUSTABLE PROSTHESIS

(71) Applicant: REHABILITATION INSTITUTE OF CHICAGO, Chicago, IL (US)

(72) Inventors: Anna Atkins, Chicago, IL (US); Shenan Hoppe-Ludwig, Chicago, IL (US)

(73) Assignee: REHABILITATION INSTITUTE OF CHICAGO, Chicago, IL (US)

(21) Appl. No.: 19/462,749

(22) Filed: Jan. 28, 2026

Related U.S. Application Data
(60) Provisional application No. 63/750,399, filed on Jan. 28, 2025.

(51) Int. Cl. A61F 2/80; A61F 2/50; A61F 2/60; A61F 2/66; A61F 2/74; A61F 2/78

(52) U.S. Cl. CPC A61F 2/80; A61F 2/60; A61F 2/748; A61F 2/7843; A61F 2002/5013; A61F 2002/502; A61F 2002/5024; A61F 2002/607; A61F 2002/6614; A61F 2002/7818

(57) ABSTRACT

An adjustable prosthetic device includes: at least one socket configured to be coupled with a prosthetic foot and defining a space for receiving a residual limb of a patient; a dilatancy pouch disposed within the rigid housing and comprising a fabric material filled with a filler material; and a sealing interface disposed onto the residual limb. The pouch is disposed over the sealing interface such that the sealing interface seals the pouch between the socket and the sealing interface. When a vacuum is applied to the pouch, the pouch becomes rigid within the volume defined between the socket and the sealing interface.

### CROSS REFERENCE TO RELATED APPLICATION

The present application claims the benefit of U.S. App. No. 63/750,399, filed Jan. 28, 2025, the disclosure of which is incorporated herein by reference in its entirety for all purposes.

### FIELD

The present disclosure relates generally to prosthetic technology. More specifically, the disclosure relates to a system and method for adjusting the socket size of a prosthesis.

### BACKGROUND

Aquatic Therapy (AT) is a beneficial and commonly used rehabilitation tool for patients with musculoskeletal, cardiopulmonary and neurologic disorders, including, post-stroke, chronic back pain, Parkinson's disease, and other neurological and musculoskeletal disorders. The physical properties of water, including density, hydrostatic pressure, buoyancy, viscosity, and thermodynamics, factor into the biological effects of AT. Buoyancy of water offloads body weight, with immersion at waist level offloading approximately 48% of body weight and 76% at the chest, increasing with greater depth. AT can facilitate greater confidence in trying new therapeutic exercises and reducing pain, allowing progression towards goals unachievable on land.

Individuals with lower limb loss or difference (LLD) may experience pain, phantom pain, decreased flexibility, reduced strength, and limited tolerance for upright activity. AT is beneficial for patients with LLD for pain reduction, encouraging weight bearing through the residual limb and offering alternative modalities to improve mobility, balance, strength, confidence, and progression to more advanced skills that may be unachievable on land. One study comparing aquatic and land therapies for 16 individuals with LLD concluded AT improved balance and exercise adaptation to a greater extent than land therapies. See, Cotrobas-Dascalu V-T, Badau D, Stoica M, et al. Impact of Kinesiotherapy and Hydrokinetic Therapy on the Rehabilitation of Balance, Gait and Functional Capacity in Patients with Lower Limb Amputation: A Pilot Study. Journal of Clinical Medicine. 2022; 11(14):4108. Yet, individuals with transtibial limb loss may be limited in participation in AT due to lack of a prosthesis for use in the pool setting.

Typically, individuals with LLD do not use a prosthesis in AT, as most daily prostheses are not often waterproof. Few individuals have a secondary custom waterproof prosthesis as they are generally not covered by insurance, making them cost prohibitive. Additionally, early in the rehabilitation phase, obtaining a prosthesis can be delayed for various reasons, including wound healing, edema or other swelling, or other complications and the individual's physical condition (e.g., strength, endurance, balance), preventing early weight bearing and gait training on land or in the water. Without a prosthesis, individuals with limb loss must balance on one leg, making entering and exiting the pool using stairs or ladders unsafe. Further, unilateral weight bearing leads to unbalanced standing posture, and dual limb exercises, including gait training and some strengthening exercises, are not possible. Providing a prosthesis to utilize in AT will reduce limitations in the pool, allowing progression of rehabilitation both on land and in the water.

The use of non-custom or interchangeable waterproof prosthetic sockets also presents challenges. Proper fit of the prosthetic socket around an individual's residual limb is crucial for patient comfort and safety. Poor fit between the residual limb and the socket can cause pain and discomfort, skin breakdowns and pressure sores, impaired mobility, balance issues, increased risk of falls, reduced functionality, discomfort during walking, and potential damage to the residual limb due to friction and uneven pressure distribution.

As such, a need exists for a non-custom prosthesis for individuals with transtibial limb difference for use in AT, that is capable of accommodating various shapes and sizes (both circumference and length) of residual limbs and that is easily adjustable for therapists, safe for the patient, and can benefit the rehabilitation experience.

### SUMMARY

Disclosed herein is an adjustable prosthesis including: a first component defining a height of the prosthesis, a second component coupled with the first component, the second component comprising a socket for receiving therein a residual limb of a patient, and a third component positioned within the socket, wherein a volume of the third component is adjustable to accommodate the residual limb. The first component is adjustable to change the height of the prosthesis and may include a pylon with a manually adjustable height component. The third component includes a dilatancy pouch filled with a filler material. The pouch may be fabricated from neoprene, and the filler material may include a plurality of expanded polystyrene beads. Other materials are contemplated. The third component may further include a sealing interface configured to seal the pouch between the socket and the sealing interface creating an airtight system, where the pouch is disposed over the sealing interface on the residual limb. Applying a vacuum to the pouch expands the pouch to occupy a volume defined between the socket and the sealing interface. In some examples, the sealing interface may be provided as a sleeve configured to be applied onto the residual limb and folded over an outer surface of the socket. The socket may include a rigid housing with a one-way valve for application of the vacuum. Each of the first component, the second component, or the third component may be made of materials suitable for use in water.

In another example, an adjustable prosthesis includes a pylon having a manually adjustable height component for changing a height of the prosthesis; a socket coupled with the pylon, the socket defining a space for receiving therein a residual limb of a patient, the socket comprising a rigid housing having a one-way valve; a dilatancy pouch disposed within the rigid housing and comprising a fabric material filled with a filler material; and a sealing interface configured to be disposed onto the residual limb. The pouch is disposed over the sealing interface such that the sealing interface is configured to seal the pouch between the socket and the sealing interface. Applying a vacuum to the pouch via the one-way valve of the rigid housing expands the pouch to occupy a volume defined between the socket and the sealing interface.

Also disclosed herein are methods for securing an adjustable prosthesis on a residual limb of a patient. The method includes disposing a sealing interface onto the residual limb; disposing a pouch over the sealing interface, the dilatancy pouch comprising a material, such as a neoprene fabric, filled with a filler material; inserting the residual limb into a socket of the prosthesis, such that the sealing interface seals the dilatancy pouch between the socket and the sealing interface; and applying a vacuum to the pouch to expand the pouch to occupy a volume defined between the socket and the sealing interface. The height of the prosthesis may also be adjusted by adjusting a height component of a pylon coupled to the socket.

The foregoing examples are just that and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the embodiments of the disclosure and are incorporated in and constitute a part of this specification, illustrate examples, and together with the description serve to explain the principles of the disclosure.

FIG. 1 is an illustration of an adjustable prosthesis according to embodiments disclosed herein;

FIGS. 2A and 2B are illustrations of elements of an adjustable prosthesis before and after vacuum is applied, according to embodiments disclosed herein;

FIGS. 3A and 3B are illustrations of elements of an adjustable prosthesis according to embodiments disclosed herein;

FIGS. 4A-4C are images of a prototype adjustable prosthesis according to embodiments disclosed herein; and

FIGS. 5A-5D are illustrations of an adjustable prosthesis according to embodiments disclosed herein.

It should be understood that some of the drawings and replicas of the photographs may not necessarily be shown to scale, or alignment perspective, unless otherwise indicated. In certain instances, details that are not necessary for an understanding of the disclosure or that render other details difficult to perceive may have been omitted. It should be understood, of course, that the disclosure is not necessarily limited to the particular examples or embodiments illustrated or depicted herein.

### DETAILED DESCRIPTION

### Definitions and Terminology

This disclosure is not meant to be read in a restrictive manner. For example, the terminology used in the application should be read broadly in the context of the meaning those in the field would attribute such terminology. Persons skilled in the art will readily appreciate that the various embodiments of the inventive concepts provided in the present disclosure can be realized by any number of methods and apparatuses configured to perform the intended functions. It should also be noted that the accompanying figures referred to herein are not necessarily drawn to scale, but may be exaggerated to illustrate various aspects of the present disclosure, and in that regard, the figures should not be construed as limiting. Some figures that represent anatomy and the positioning of embodiments relative to that anatomy should be interpreted with some deviation permitted as the anatomical structures depicted will vary in size and position from person to person.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Z₀, the phrase is intended to refer to a single element selected from X, Y, and Z, a combination of elements selected from the same class (e.g., X₁ and X₂) as well as a combination of elements selected from two or more classes (e.g., Y₁ and Z₀).

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

### DESCRIPTION OF VARIOUS EMBODIMENTS

The present disclosure relates to devices and methods for adjusting the socket size of a prosthesis. Utilizing the principle of dilatancy, the socket of a prosthesis may be adjusted to fit most individuals with transtibial limb difference, for example, for use in aquatic therapy. Dilatancy is a principle according to which a substance rigidity increases with pressure. With vacuum applied in a closed system, small particles can hold a shape-imagine a vacuum-sealed bag of coffee beans. While dilatancy has been utilized in prosthetics through the CIR casting system for creating transtibial sockets, dilatancy has not been applied within a socket to provide an accommodative shape for multiple users or a single user experiencing changing residual limb shape or size.

The adjustable prosthesis described herein may be provided as a customizable transtibial prosthesis system, comprising of a set of interchangeable sockets and pylons, and an inner-socket adjustment element. This system may be provided in one or more sizes (e.g., small, medium and large) and may be adjustable in height, residual limb length, and socket volume to accommodate various residual limb presentations. Physical therapists may use this tool in an aquatic environment to perform tasks such as safe entering and exiting the pool via stairs, independent standing with weight bearing through the residual limb, flutter kicks, and strength-based activities. Applications of this invention include but are not limited to: use in aquatic therapy centers and rehabilitation centers for aquatic therapy. Other applications are contemplated.

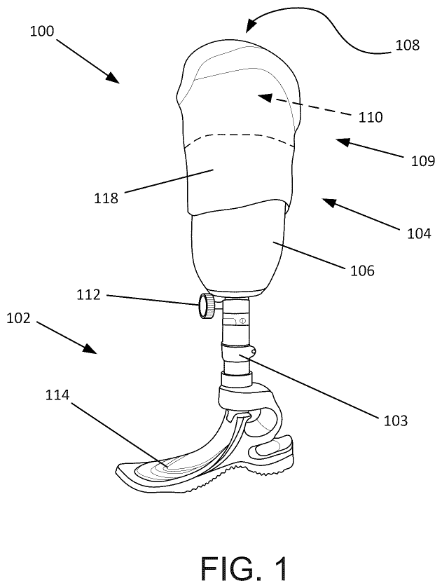

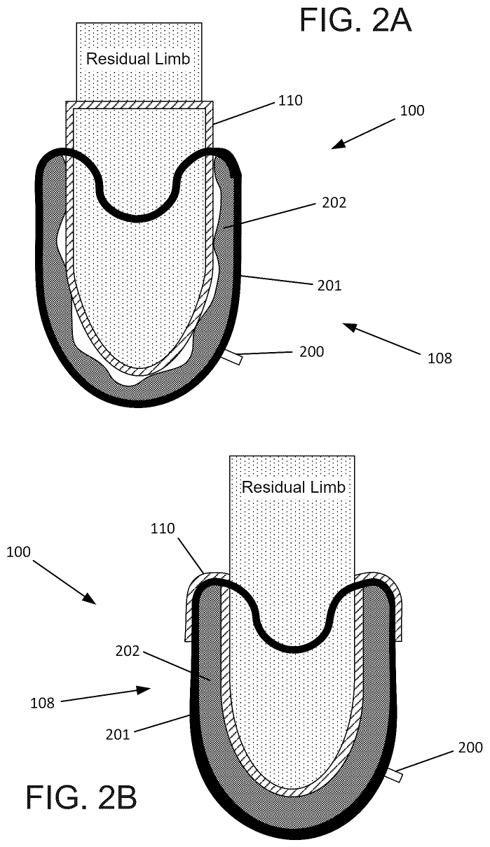

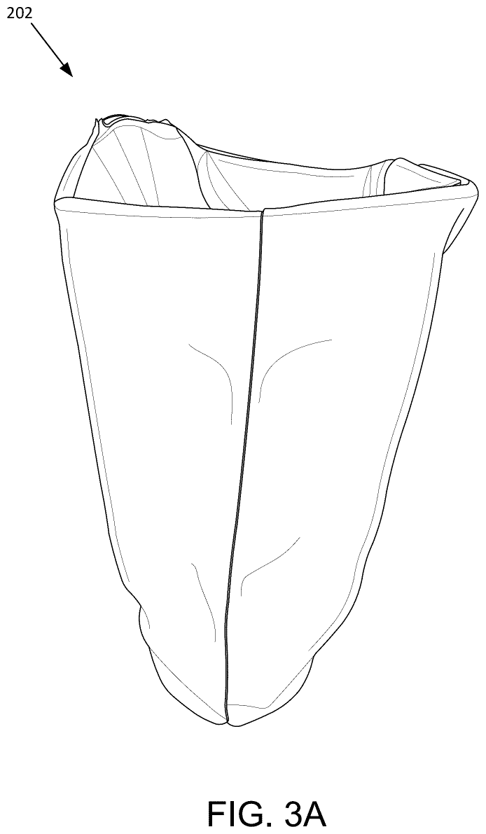

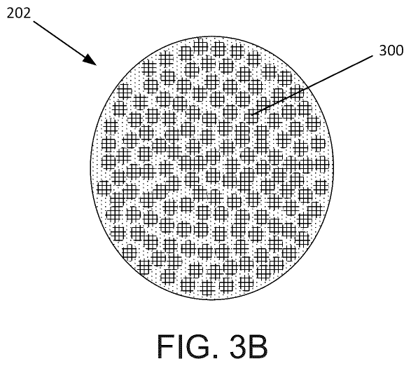

FIG. 1 illustrates an example of an adjustable prosthesis 100 or adjustable prosthetic device according to embodiments disclosed herein. The prosthesis 100 includes a first component 102 defining a height of the prosthesis 100, a second component 104 coupled with the first component 102 that includes a socket 106 for receiving therein a residual limb of a patient, and a third component 108 positioned within the socket 106. A rigidity of the third component 108 is adjustable to accommodate the residual limb. The first component 102 may include a pylon 103, which may be adjustable in height, an adapter 112 for connecting pylon 103 to socket 106, and a terminal prosthetic 114 (such as a prosthetic foot 114). In one embodiment, as shown in FIGS. 2A and 2B, the socket 106 includes a valve 200 disposed in a rigid housing 201. The third component 108 (also referred to herein as the socket adjustment element) includes a sealing interface 109 (such as a liner 110) and a dilatancy pouch 202. A suspension sleeve 118 may be positioned over the outside of the socket 106 and reflected proximally onto the thigh to provide a secure attachment between the prosthesis 100 and the user. A separate vacuum pump (not shown) may be used to apply vacuum to the dilatancy pouch 202 via the valve 200, which may be a one-way or unidirectional valve. An example of the dilatancy pouch 202 is also shown in FIG. 3A. In some examples, a plurality of sizes of dilatancy pouches 202 may be used in the prosthesis 100. The dilatancy pouch 202 may be constructed of a fabric, such as neoprene, and may be filled with a filler material 300, such as expanded polystyrene (EPS) beads, as shown in FIG. 3B. In some examples, the EPS beads may be between 2 mm and 3 mm in diameter. Other materials may be used, including non-expanded polystyrene (NEPS) beads, buckwheat hulls, silica sand, vermiculite and combinations of multiple materials. Table 1 summarizes ranked rigidity under vacuum and weight of various filler materials. Rigidity is ranked on a scale of 1-8, where 1 is most rigid and lightest. EPS material may be used for increased rigidity and reduced weight to facilitate socket comfort and control. Additional EPS thickness at the distal tibia and triangulation of the socket 106 improved comfort based on trialing with individuals with LLD in two fittings.

TABLE 1
Ranking rigidity under vacuum and weight of materials.
Ranking of 1-8, where 1 is most rigid and lightest.

| Material | Rigidity | Weight |
|---|---|---|
| Sand | 1 | 8 |
| Vermiculite* | 2 | 2 |
| Expanded Polystyrene | 3 | 1 |
| 20% Sand/80% Vermiculite | 4 | 4 |
| 50% Sand/50% NEPS | 5 | 7 |
| 20% Sand/80% NEPS | 6 | 6 |
| NEPS | 7 | 5 |
| Buckwheat Hulls | 8 | 3 |

*Vermiculite displayed high rigidity and light weight; however, it is not safe to be exposed to skin or inhaled.

The sealing interface 109 is configured to create an airtight system between the socket 106 and the portion of the sealing interface 109 in contact with the residual limb, enclosing the dilatancy pouch 202, allowing air to be removed. The sealing interface 109 may be formed of any airtight and elastic material. In one example, the sealing interface 109 is provided as a prosthetic liner 110 that is fitted over the residual limb and then folded over the socket 106, thereby sealing the dilatancy pouch 202 between the socket and the liner.

Additionally, the first component 102 may also include an adjustable height element, such as an adjustable height pylon 103. The pylon 103 may be manually adjustable, using a telescoping tubing system, for example. Additionally, the transverse rotation of the pylon may also be adjustable. The pylon 103 may be connected to a terminal prosthetic 114. For transtibial prostheses, the terminal prosthetic 114 may be provided as a prosthetic foot attached distal of the adjustable pylon 103. An adapter 112 may be used to connect socket 106 to pylon 103 or directly to the terminal prosthetic 114.

In operation, the sealing interface 109, which may include the liner 110, is first placed onto a residual limb. The dilatancy pouch 202 is then placed on the residual limb, over the sealing interface 109 (e.g., liner 110). The rigid socket 106 is then fitted over the dilatancy pouch 202 (or, alternatively, the dilatancy pouch 202 is disposed inside the socket 106). In some cases, additional comfort elements, such as pre-tibial pads or gel spots may be placed as needed within the socket 106 to relieve discomfort at pressure points identified by the wearer or clinician. Distal end pouches may also be inserted into the socket for adjusting to different residual limb heights. Next, the dilatancy pouch 202 is sealed between the socket 106 and the sealing interface 109 (e.g., liner 110). Where the sealing interface 109 is provided as a liner 110, it is reflected down (folded) onto the rigid socket as shown in FIG. 2B, which creates a closed, airtight system between the socket and the portion of the sealing interface 109 in contact with the residual limb. At this stage, once the residual limb, with liner 110 and dilatancy pouch 202 disposed thereon, are all placed within the socket 106 and sealed, the wearer may transition to standing to ensure proper positioning of the socket 106 on the residual limb as shown in FIG. 2A. Vacuum is pulled onto the dilatancy pouch via the one-way valve in the socket using an external vacuum pump. As vacuum is applied, air is removed from the closed, airtight system, as shown in FIG. 2B, causing the materials sealed between the portion of the sealing interface 109 in contact with the residual limb and the socket 106—e.g., the dilatancy pouch 202 and the filler material 300—to become rigid, forming a custom rigid socket 106, as seen in FIG. 2B, around the residual limb due to the principle of dilatancy. Application of vacuum transitions the pouch 202 from a soft pouch to a more rigid pouch. The rigidity achieved by applying vacuum to the dilatancy pouch 202 is dependent on the type of filler chosen (different fillers create different rigidities, see Table 1) and the pouch material thickness, amount of elasticity and tightness of the weave (how effectively vacuum can be applied to the filler material). The vacuum applied to the pouch 202 causes the pouch 202 to become rigid within the volume defined between the socket 106 and the sealing interface 109 (e.g., liner 110).

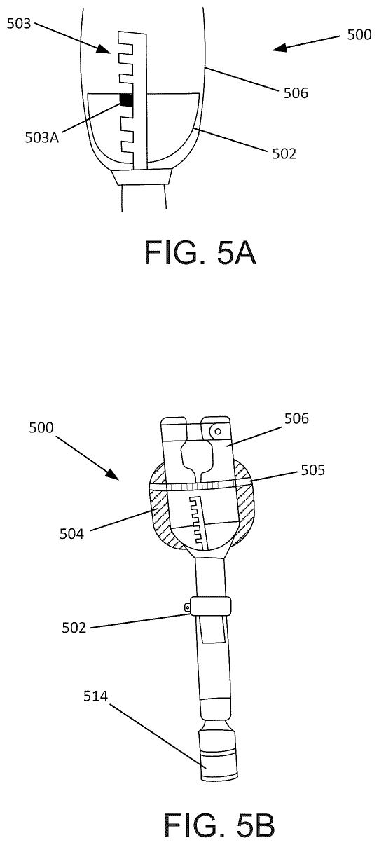

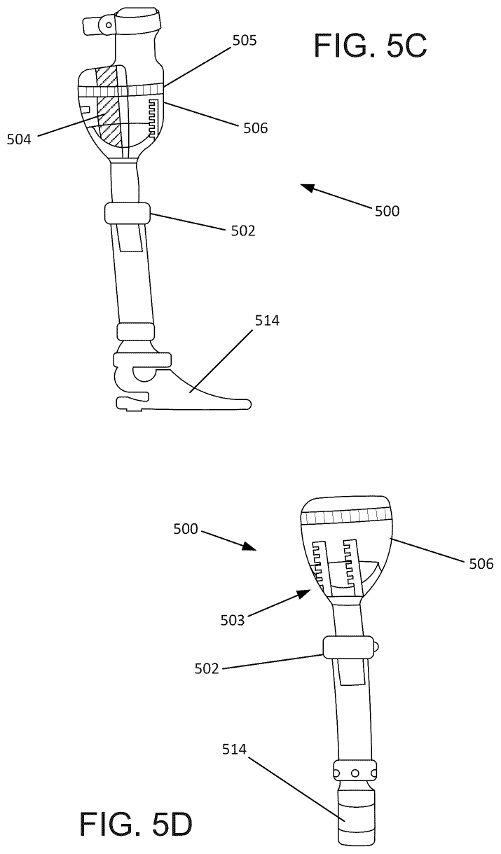

FIGS. 5A-5D illustrate another adjustable prosthesis 500, according to some examples. FIG. 5A shows the prosthetic sleeve according to some examples. The prosthesis 500 may include a distal end cup 502 within the socket 506 for additional height adjustment. For example, the cup 502 may be coupled with a manually adjustable height component, such as an adjuster 503, that has a plurality of teeth or edges, where each tooth or edge defines a predetermined height for the location of the cup 502. When the cup 502 is coupled at a certain tooth or edge (e.g., the shaded tooth 503A) using a fastener, the cup 502 is configured to hold the residual limb at the height as determined by the tooth or edge 503A. FIG. 5B shows a view of an adjustable prosthesis according to some examples. The prosthesis 500 may include an overlap component 504 disposed at the socket 506. The overlap component 504 may be made of polymer such as polyethylene to provide additional support. The overlap component 504 may be held in place with respect to the socket 106 using a fastener 505 such as a buckle clip. FIG. 5C shows another view of the adjustable prosthesis of FIG. 5B. FIG. 5D shows another view of the adjustable prosthesis of FIG. 5B without the overlap component 504. More than one adjuster 503 may be implemented, as shown in FIG. 5D.

### Experimental Example

A study exploring the development and usability of an adjustable prosthesis, utilizing dilatancy, for aquatic therapy was conducted.

Methods: Participants underwent an informed written consent process (IRB STU00219525). Three individuals (1M 55 yrs and 2F 47, 22 yrs) with unilateral transtibial amputations were recruited. A set of interchangeable adjustable sockets and pylons were developed. Utilizing dilatant properties, small particles under vacuum were assessed for their ability to provide a rigid, lightweight, and re-moldable pouch within the socket. Participants trialed the prosthesis during two fittings and three aquatic therapy sessions. Upon completion, participants and treating therapists completed the System Usability Scale. Survey results were converted to a 100-point scale. Time to donn the prosthesis was recorded and socket comfort scores at the final session were averaged.

Results: The System Usability Scale indicated that the prosthesis was usable for aquatic therapy. Participant scores were 97.5, 95, and 87.5, and therapist's scores were 92.5 and 85 of a total possible 100. Average socket comfort scores were 10, 9, and 7.6/10. Times to donn the prosthesis were 5:45, 5:32, and 4:10 (m: sec).

Conclusions: This system functioned successfully as a non-custom prosthesis for multiple users in aquatic therapy, from the participant and therapists' perspective.

Clinical Significance: The novelty of this system is utilizing a prosthesis for multiple users, improving the rehabilitation capabilities during aquatic therapy.

STUDY DESIGN AND PARTICIPANT RECRUITMENT: An adjustable prosthesis system was developed and tested on three individuals during two fitting and three AT sessions. Participants were recruited from Shirley Ryan AbilityLab following recommendation from a clinical prosthetist or physical therapist (PT) for meeting inclusion criteria and appropriate to test a prototype system. Inclusion criteria was 18-89 years of age, unilateral transtibial limb difference (TTLD), K2-K4 level, ability to wear a prosthesis, and active in AT, recently completed within six-months, or determined a good candidate for AT by a therapist. Exclusion criteria included comorbidities which would interfere with the study design, pregnancy, contraindications for AT, and inability to walk without an assistive device for two-minutes. Written consent of participants was obtained prior to study involvement. All participants were active in PT at consent, and two had completed at least one clinical AT session prior to consent. Table 2 summarizes demographic and adjustable prosthesis information for each participant.

TABLE 2
Participant demographics at time of study enrollment.

| | | ID | | |
|---|---|---|---|---|
| | | 1 | 2 | 3 |
| Age (yrs) | | 47 | 55 | 22 |
| Gender | | F | M | F |
| Height (m) | | 1.69 | 1.8 | 1.67 |
| Body Mass (kg) | | 63 | 95.5 | 95.2 |
| Cause of Amp. | | Dysv | Dysv | Blood Clot |
| Amp. Laterality | | Right | Left | Right |
| Yrs. Since Amp. | | 3.5 | 2 | <1 |
| K-Level | | 3 | 3 | 3 |
| Suspension | | Locking | Locking | Suction |
| Adjustable | Socket and | M | S | L |
| Prosthesis | Pouch | | | |
| Sizing | Pylon | Tall, 11 mm | Tall, max | Short, min |
| | | extended | height | height |
| | Distal end Pad | 3 cm | 2 cm | 3 cm |
| | Pre-tibial pads | Pre-tibial pads | None | Gel Spots |
| | or gel spots | | | |

(Amp. = Amputation, Dysv = Dysvascular, Sizing S/M/L = Small, Medium, Large).

Two physical therapists provided initial insight on desired features of the prosthesis and administered AT, alternating sessions due to availability. At the conclusion of the study, their feedback was obtained after consent.

DILATANCY: To identify the optimal material for the adjustable dilatancy design, particles were filled into neoprene pouches, placed inside a plastic bag, and vacuum was pulled to allow ranking of rigidity under vacuum. Additionally, a consistent volume of the materials was weighed for ranking. Materials tested included: non-expanded polystyrene (NEPS), expanded polystyrene (EPS), buckwheat hulls, silica sand, and vermiculite. Materials were selected with the following reasoning: EPS and buckwheat are commonly used in bean bag chairs, NEPS due to its small, uniform shape, silica sand and vermiculate for their availability in prosthetic environments. A neoprene pouch was filled with the optimal material and used to cover the residual limb (RL).

SOCKET DESIGN AND FABRICATION (FIG. 1): Prosthetic socket design and componentry were chosen to allow independent PT donning with minimal need for adjustments. The socket 106 (e.g., selectable from one of 3 or more different sizes) consisted of thermoformed Vivak® with a 4-hole plate at the distal end and a one-way valve. A prosthetic liner 110, such as an Alps® Skin Reliever Liner, served as the interface between the RL and dilatancy pouch. Any sufficiently elastic liner, for example a thermoplastic elastomer (TPE) gel liner or silicone liner, can be used as the interface. The liner 110 was reflected outward onto the socket 106 (i.e., folded over the outside of the socket), creating a closed system for the dilatancy pouch. Inside the socket 106 was a custom fabricated one-millimeter double layer neoprene dilatancy pouch 202 (e.g., selectable from one of 3 or more different sizes) filled with two-to-three-millimeter diameter EPS, as part of the third component 108. The socket 106 was suspended to the RL with the sleeve 118. Distal to the socket 106, an adapter 112 (e.g., the Xtend® by Lindhe four-hole-plate) facilitated swapping sockets 106 to adjustable pylons 103, as part of the first component 102. First component 102 may comprise various combinations or arrangements of elements to accommodate all patient heights. The combinations included attachment of the socket directly to the prosthetic foot, or attachment of a pylon to foot, using an adjustable height pylon (such as those sold by Ossur). The height ranges from mid-patellar tendon (MPT) to floor accommodated were 35.4 cm to 51.9 cm. The Niagara Foot™ by Empire Medical, a universal 26 cm Hydex® plastic foot, was used with a 1.2 cm heel wedge and rubber treading adhered to the plantar surface. Any appropriate terminal prosthetic 114 could be used. Socket alignment included 5° extension (e.g., with respect to a sagittal direction or plane), 0° adduction/abduction (e.g., with respect to the coronal and transverse directions or planes), and no inset/outset. In the transverse plane, the foot was rotated to the appropriate toe out when setting pylon height. Socket sizing was guided by average anthropomorphic lower leg lengths and typical transtibial amputation lengths. The expected MPT circumferences to be accommodated were 20-44 cm and RL lengths 8-23 cm.

The rigid socket shape was relatively cylindrical with mild anterior triangulation. Additional hook-and-loop (e.g., Velcro®) pre-tibial pads were used for loading the RL as needed. Gel spots were used under the liner for pressure-point relief as needed.

To donn the prosthesis 100, a prosthetic liner 110 was first applied to the RL, and over this, the neoprene pouch with EPS was donned. EPS was shifted around the RL, covering bony prominences as needed. Distal end pads with EPS accommodated height from MTP to distal end of the socket, with appropriate fit determined with the patella sitting just proximal to the anterior trimline. The socket 106 was donned and then the liner 110 was reflected to the outside of the socket 106, sealing the pouch from the RL. A suspension sleeve 118 was donned over the outside of the prosthetic socket 106 and was reflected proximally onto the thigh to provide a secure attachment between the prosthesis 100 and the user and to prevent the prosthesis 100 from sliding off the user's leg. Vacutec™ 800 EV2 Aspirator pump was attached to the one-way valve, and 203-254 mmHg vacuum was applied (FIGS. 2A and 2B), which is below the typical levels used in elevated vacuum prosthetic applications. In some examples, the sleeve 118 is disposed or wrapped around the portion of the liner 110 that is folded over an outer surface of the socket 106 so as to hold the liner 110 in place with respect to the socket 106, as shown in FIG. 1.

FITTING AND THERAPY VISITS: Participants attended two fitting visits with prosthetic team members for development of the prosthesis and appropriate sizing; standard clinical practice principles of static and dynamic alignment were applied with a goal of successful therapeutic use in AT. Sizing was recorded and replicated for all AT visits. The sizing consisted of socket and corresponding dilatancy pouch size, pylon length, distal end pouches used, and any necessary gel spots and/or pre-tibial pads. Two PTs were trained to donn the prosthesis. Each participant completed three thirty-minute AT sessions led by a PT, completing exercises, gait training, and swimming. During initial AT sessions, prosthetists assisted the therapist in prosthesis donning. At each participant's third session, the PT independently donned it.

OUTCOMES: The primary outcome was the System Usability Scale (SUS), administered to participants at the conclusion of their third AT session. The two PTs also completed the SUS at the end of the participant sessions. The SUS is a 10-item questionnaire assessing usability of a variety of items from the consumer's perspective. This survey is not specific to prosthetic devices, but universally used with strong validation. Survey results were converted to a 100-point scale.

Secondary outcomes included socket comfort scores (SCS) and timed prosthesis donning by the therapist at the third AT session. SCS is a 0-10 ranking of comfort in increasing order. Each participant's SCS from the third AT session at donning, during AT, and after AT were averaged.

### Results

DILATANCY MATERIALS: EPS displayed an optimal combination of rigidity and weight and was selected for use in the dilatancy pouches. Other materials were rejected for a variety of reasons: vermiculite demonstrated high rigidity and low weight, however, was excluded due to the potential for asbestos contamination; silica sand was difficult to disperse around the RL; NEPS pulled through seams of the sewn pouch; and buckwheat hulls were not comfortable within the pouch. Table 1, above, summarizes these findings.

OUTCOMES: Participant's SUS scores were 97.5, 95, and 87.5 of the total 100 possible points. Therapist's scores were 92.5 and 85. Average SCS during the third session were 10, 9, and 7.6/10, as summarized in Table 3. Recorded times for the therapist to donn the prosthesis were 5:32, 5:45 and 4:10 (m: sec).

TABLE 3
Socket comfort score before, during, and after the third aquatic
therapy session for each participant.

| ID | Pre | During | Post |
|---|---|---|---|
| 1 | 7 | 9 | 7 |
| 2 | 10 | 10 | 10 |
| 3 | 8 | 10 | 9 |

### DISCUSSION

The Veterans Administration Clinical Practice Guidelines (CPG) recommends appropriate equipment and assistive technology be provided during the perioperative stage of amputation and emphasizes importance of early mobility training after amputation, including weight bearing through the RL. An adjustable prosthesis for AT could assist in meeting both CPG recommendations for individuals with TTLD. This study was the initial work developing and assessing the usability of an adjustable prosthesis to allow early gait training in AT, enhancing the rehabilitation benefit for individuals with LLD.

DILATANCY: Dilatancy properties of EPS offered an adjustable socket with total contact for multiple individuals. The inner shape conformed and hardened to the RL, offloading the distal tibia and translating forces to the prosthesis for actuation. It was determined that material to fill the dilatancy pouch should be lightweight and rigid under vacuum, for socket comfort and control. EPS provided an optimal combination of these characteristics. Similar strategies with EPS are used for custom wheelchair impressions.

FITTING SESSIONS: Fitting sessions provided feedback on the design and guided sizing recommendations. Participants noted improved comfort when vacuum was applied to the pouch in standing, rather than seated or offloading the RL. The greatest comfort was achieved with 203-254 mmHg vacuum. Participants reported 127 mmHg did not provide enough relief to the distal RL, and higher levels near 381 mmHg resulted in the socket feeling tight. This vacuum technique is different from traditional elevated vacuum (EV) applications. EV is a method of prosthesis suspension where a mechanism or pump pulls the air out of the prosthetic socket, generating a negative pressure that pulls a liner and the wearer's RL towards the inner socket wall. In the present system, rather than applying vacuum to the entire system, pulling the RL towards the inner socket walls as in EV, the non-custom sockets using dilatancy only apply vacuum to the dilatancy pouch. Due to this, lower levels of vacuum resulted in appropriate socket comfort and fit. Initial socket triangulation provided appropriate offloading of the distal tibia for two participants, while the third required additional pretibial pads added via hook-and-loop closure. One participant required a gel spot placed over a sensitive area which relieved discomfort.

Prosthetic alignment goals for the system included a safe and stable gait for both left and right use. Neutral coronal alignment resulted in stable gait, with minor varus or valgus at mid-stance on land, eliminated in the water. Participants denied discomfort resulting from this deviation. The socket was initially set in standard bench alignment of 5° sagittal flexion, resulting in excessive knee flexion at initial contact. For all three participants, 5° of extension eliminated this deviation and improved participant comfort.

It was expected that the distal end pads would accommodate the difference between RL length and socket length. This was not found to be consistent, most likely due to the large amount of variation in the placement of the EPS within the pouch based on limb shape. More EPS located at the distal end required less distal pads. For all three participants, less distal pads were required than expected. Participant's distal end pad sizing was consistent in each AT session, without issue. The sizing of distal end pads will require further investigation to create a sizing guide.

The initial concept for this prosthesis was to not require the input of a prosthetist for sizing selection or fitting, however, it is expected that an initial evaluation by a prosthetist will be required prior to a therapist donning. It is expected that this will improve the participant's socket comfort, reduce time required by the PT, and verify appropriateness of the device.

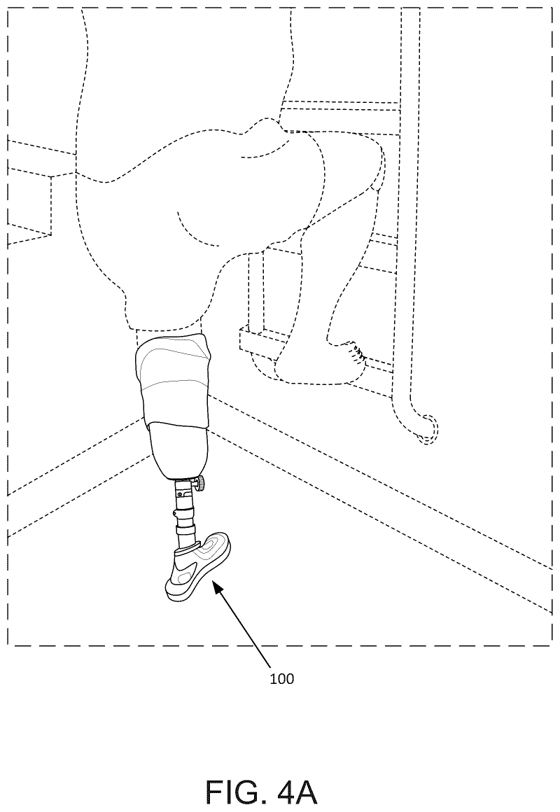

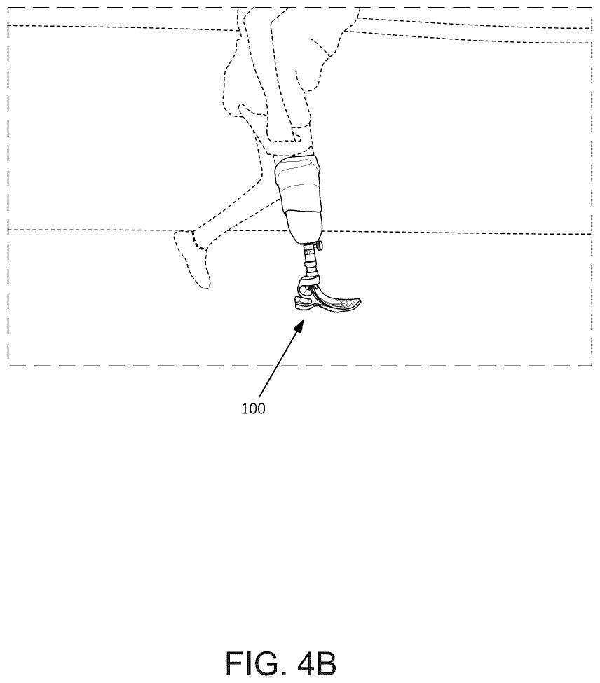

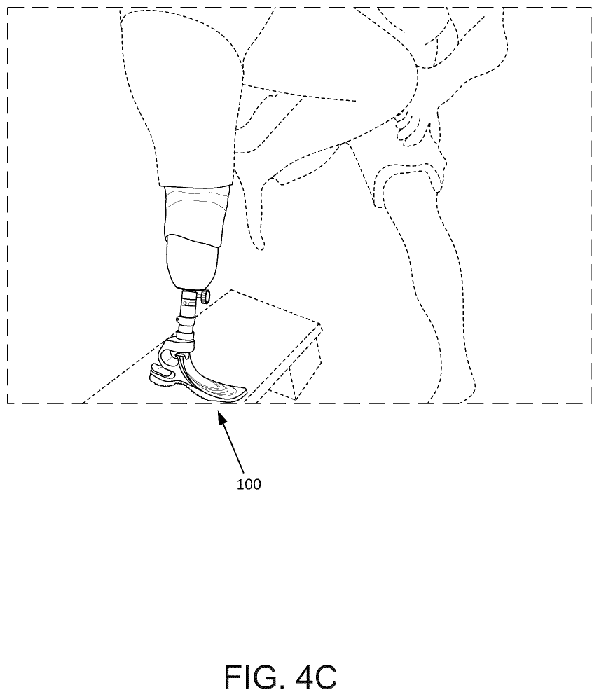

AT SESSIONS: FIGS. 4A-4C illustrate the adjustable prosthetic device 100 donned by a patient and in use in aquatic therapy. Both therapists facilitating AT sessions frequently treat individuals who use prostheses. One therapist led four AT sessions, and the other led five sessions. With the prosthesis 100 donned, participants entered the pool via stairs or a ladder (FIG. 4A), then engaged in gait training (FIG. 4B), strengthening (not shown) and balance (FIG. 4C) exercises, and swimming, with the RL weight bearing, stabilizing, or as a longer lever for strength activities. Occasionally, hydro-tone fins were attached to the prosthesis for added resistance.

Resisted side steps induced minor discomfort for one participant at the medial proximal socket trim line. It is speculated that discomfort occurred due to the pouch shifting during donning of the prosthesis or insufficient pouch height at the femoral condyles. The pouch height may be increased to address the discomfort. Additionally, one participant experienced slight movement of the prosthesis on the RL while swimming breaststroke. This distraction may be due to the force on the prosthesis while swimming and shifting of proximal thigh tissues when kicking, magnified by the high elasticity of the suspension sleeve. All participants experienced minor amounts of water entering through the proximal aspect of the suspension sleeve, however the motion or water in the sleeve did not impact suspension, comfort, or function.

SYSTEM USABILITY SCALE: The participants and the therapists completed the SUS as they are both ‘users’ from a unique perspective. Participant feedback demonstrates clinical interest from a patient perspective, while therapist feedback will guide the potential use in AT, as a PT will be required for fitting and using the system.

Participant SUS scores of 97.5, 95, and 87.5 and PT scores of 92.5 and 85 demonstrate a usable system with the grading of ‘A+’ or greater than passable for usability based on recommended scoring. Table 4 sets out participant and therapist SUS scores. Compared to over 5000 SUS scores assessing a variety of systems, our results fall into the 96-100 percentile range demonstrating users are more likely to recommend this to others. Questions which assess the likelihood of using the system frequently and confidence using the system, resulted in participants selecting maximum values. All participants deducted a point when evaluating their required technical support to use the system, and one participant deducted a point when assessing the ease of use of the system. Our goal is for the prosthesis to be easily adjusted by the therapist, and not intended for the participant to independently donn the prosthesis. Due to this, the participant responses are acceptable. An important element is the learnability of the system from the therapist perspective. Both therapists reported that they ‘strongly disagree’ with needing support from a technical person to use the system. These responses reflect that after initial training in the system, therapists may be able to operate it independently for AT sessions.

SOCKET COMFORT SCORES: During the fitting sessions, reported SCS were lower than scores reported at the final AT session. This is attributed to the optional additional removable pre-tibial pads and gel spots. It's important to note, two participants had slightly lower SCS on land (7-8/10) than in the water (9-10/10). This is expected as body weight is offloaded with water immersion. The lowest SCS reported while in the pool was 9/10, demonstrating a high degree of comfort during all therapy activities in the pool, even when loading forces were increased during jumping and resistance training.

TIME TO DONN: Donning time is expected to be reduced with therapist repetition. Times of 5:32 and 5:45 (m: sec) were recorded by separate therapists at the final AT session for two participants. At the last participant's final AT session, the therapist had gained greater experience donning the prosthesis by this time, and 4:10 (m: sec) was recorded. Additionally, 60-90 seconds were included for allowing the vacuum pump to pull air from the system. This can likely be reduced, as extra time was allotted for beginning trials. An additional time intensive step was reflecting the sleeve onto the thigh. This could be simplified with assistance from the participants but was not allowed for consistency between participants. Additionally, alternative sleeve designs could be considered.

LIMITATIONS, ATTEMPTED MITIGATIONS, AND FUTURE RESEARCH: Participants represented a sample of prosthetic users, and the design should be further assessed by a population with greater variability in age, K-levels, RL presentation, and time since amputation. It is important to note the relatively narrow RL size variance tested, with circumferences at MPT measuring 32-40 cm and RL length 14-16 cm.

PTs conducting AT in this study also completed the SUS to evaluate the feasibility of this design in a clinical setting which may induce bias. However, this prototype was not used outside of the study, and it was important to record the therapist's initial perspective on the system. Reviews from alternative PTs will be obtained in the future.

CONCLUSION: This system functioned successfully as a non-custom prosthesis for multiple users in AT and was determined as useful from both the participant and therapists' perspectives. Applying vacuum to EPS, within a neoprene pouch, created a rigid interface that was successfully used on multiple RL, allowing a therapist to conduct gait training, bilateral balance training, and other dual limb activities not typically accessible to individuals with LLD in AT.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

What is claimed is:

1. An adjustable prosthesis comprising:
a first component defining a height of the prosthesis;
a second component coupled with the first component, the second component comprising a socket for receiving therein a residual limb of a patient; and
a third component positioned within the socket, wherein a volume of the third component is adjustable to accommodate the residual limb.

2. The prosthesis of claim 1, wherein the first component is adjustable to change the height of the prosthesis.

3. The prosthesis of claim 2, wherein the first component includes a pylon with a manually adjustable height component.

4. The prosthesis of claim 1, wherein the third component includes a dilatancy pouch filled with a filler material.

5. The prosthesis of claim 4, wherein the dilatancy pouch is fabricated from neoprene, and the filler material includes a plurality of expanded polystyrene beads.

6. The prosthesis of claim 4, wherein the socket includes a rigid housing, and the pouch is disposed within the rigid housing of the socket.

7. The prosthesis of claim 6, wherein the third component includes a sealing interface configured to seal the pouch between the socket and the sealing interface.

8. The prosthesis of claim 7, wherein the sealing interface is a sleeve configured to be disposed onto the residual limb and folded over an outer surface of the socket.

9. The prosthesis of claim 7, wherein applying a vacuum to the pouch expands the pouch to occupy a volume defined between the socket and the sealing interface.

10. The prosthesis of claim 9, wherein the rigid housing of the socket includes a one-way valve, and the vacuum is applied via the one-way valve.

11. The prosthesis of claim 1, wherein any one or more of the first component, the second component, or the third component are made of materials suitable for aquatic therapy.

12. An adjustable prosthesis comprising:
a pylon having a manually adjustable height component for changing a height of the prosthesis;
a socket coupled with the pylon, the socket defining a space for receiving therein a residual limb of a patient, the socket comprising a rigid housing having a one-way valve;
a dilatancy pouch disposed within the rigid housing and comprising a fabric material filled with a filler material; and
a sealing interface configured to be disposed onto the residual limb and, wherein the pouch is disposed over the sealing interface such that the sealing interface is configured to seal the pouch between the socket and the sealing interface;
wherein, when a vacuum is applied to the pouch via the one-way valve of the rigid housing, the pouch expands to occupy a volume defined between the socket and the sealing interface.

13. The prosthesis of claim 12, wherein the fabric material is neoprene, and the filler material includes a plurality of expanded polystyrene beads.

14. The prosthesis of claim 12, wherein the pylon, the socket, and the sealing interface are made of materials suitable for aquatic therapy.

15. A method of securing an adjustable prosthesis on a residual limb of a patient for aquatic therapy, the method comprising:
disposing a sealing interface onto the residual limb;
disposing a pouch over the sealing interface, the pouch comprising a fabric material filled with a filler material;
inserting the residual limb into a socket of the prosthesis, such that the sealing interface seals the pouch between the socket and the sealing interface; and
applying a vacuum to the pouch to expand the pouch to occupy a volume defined between the socket and the sealing interface.

16. The method of claim 15, further comprising:
adjusting a height of the prosthesis by adjusting a height component of a pylon coupled to the socket.

17. The method of claim 15, wherein the fabric material is neoprene, and the filler material includes a plurality of expanded polystyrene beads.
